(19) United States
(12) Patent Application Publication
(10) Pub. No.: US 2003/0207283 A1
(43) Pub. Date: Nov. 6, 2003

(54) Isolated polynucleotide encoding a novel phosphate transporter in plants and a method of modulating phosphate uptake in plants

(76) Inventor: Avraham Levy, Rehovot (IL)

Correspondence Address: Anthony Castorina, G E Erlich (1995) Ltd, Suite 207, 2001 Jefferson Davis Highway, Arlington, VA, 22202, US

(21) Appl. No.: 10/181,037

(22) PCT Filed: Jan. 26, 2001
(86) PCT No.: PCT/IL01/00075

(30) Foreign Application Priority Data
Jan. 27, 2000 (IL) 134258

(51) Int. Cl. C12Q 1/68; C07H 21/04; C12P 21/02; C12N 5/06; C07K 14/47

(52) U.S. Cl. 435/6; 435/69.1; 435/320.1; 435/325; 530/350; 536/23.5

(57) ABSTRACT

An isolated polynucleotide encoding a novel plant phosphate transporter and, more particularly, to an essential phosphate transporter which, when inactivated, is associated with phosphate deficiency syndrome in plants, and to methods for using same to modulate, i.e., increase or decrease, phosphate uptake in plants.

### FIELD AND BACKGROUND OF THE INVENTION

The present invention relates to an isolated polynucleotide encoding a novel plant phosphate transporter and, more particularly, to an essential phosphate transporter which, when inactivated, is associated with phosphate deficiency syndrome in plants, and to methods for using same to modulate, i.e., increase or decrease, phosphate uptake in plants.

In modem commercial agriculture, plants are grown at high density, and as such profitability depends upon both length of production cycle and yield per unit area. In many cases, the amount of nutrients available in the soil is insufficient to facilitate production consistent with these profitability constraints. Therefore, fertilizers which supplement the nutrients found in soil are commonly utilized by growers. Commonly used fertilizers typically contain nitrogen and phosphate as their primary ingredients and are a substantial part of the production cost in plant agriculture. The cost of phosphate fertilizers (Pi) Worldwide is several billion dollars annually and although this cost is justified by increased profitability, other considerations make it desirable to limit the use of phosphate fertilizers. In 1993, US farmers used about 4.45 million tons of Pi fertilizers valued over a billion dollars. In view of the high costs involved in fertilizer application, there is a need to improve the ability of plants to acquire nutrients from the soil.

Phosphate fertilizers are generally obtained from non-renewable sources and at the current rate of usage, it is estimated that the available supply will last only 60 to 90 years. Moreover, phosphate fertilizers are one of the major polluting factors in the aquifer. In view of the high costs involved in fertilizer application, and of the fact that Pi is a non-renewable resource and of the environmental damages caused by excess Pi fertilization, there is a need to improve the ability of plants to acquire Pi from the soil.

At present, even under the best agronomic conditions, there are physiologic and genetic limitations for enhancing crop yield above a certain level. It is therefore necessary to develop input efficient plants.

Phosphorus is one of the least available plant nutrients in the soil. Phosphate (Pi) deficiency is widespread in nature and due to the interactions of Pi with soil particles, up to 80% of the applied phosphates may be fixed, forcing growers to apply 4 to 5 times the amount of fertilizer theoretically required by the crop.

Pi acquisition in plants is regulated by two key soil and plant factors: Pi availability in the rhizosphere and ability of the plants to acquire available Pi. For a recent review on phosphate acquisition in plants, see, K. G. Raghothama, 1999, Proc. Natl. Acad. Sci., USA, May 11; 96(10):5868-5872).

Plants have developed elegant adaptive mechanisms to enhance both the availability and uptake of Pi. Under low Pi availability, the expression of a high affinity Pi transporter is upregulated, so as to increase Pi acquisition by roots. Pi is transported by phosphate transporters in an energy mediated co-transport process, driven by protons generated by a plasma membrane H⁺ ATPase. Pi absorption is accompanied by H⁺ influx with a stoichiometry of 2 to 4H⁺/H₂PO₄ transported.

In addition to phosphate transporter upregulation, plants also produce and secrete enzymes such as phosphatases in response to Pi deficiency. It is presumed that phosphatases released to the rhizosphere aid in the release of Pi from organic phosphorus compounds.

Tomato as a Gelletic Model to Understand Phosphate Nutrition:

The total consumption of phosphate fertilizers to grow tomato in the USA was approximately 55 million pounds in 1994. Nearly 60% of all the phosphate fertilizers were used in the state of California. The tomato market is valued at tens of billions of dollars annually. Besides its significant position in the World vegetable market tomato is used as a genetic and molecular model for crop improvements. The recent developments in genomics have made tomato one of the economically important model crops for genetic improvements. Development of plants that are efficient in obtaining phosphorus is critical for the advancement of sustainable agriculture.

Worldwide, tomato is one of the most important crops in the fresh vegetable market as well as in the food processing industry (Rick and Yoder, 1988). The yearly world production of processed tomatoes exceeds 26 million tons with California producing over 49% of world supply. In addition to being one of the commercially important vegetable crops, tomato is a model plant for molecular manipulations and breeding experiments. It has a relatively small diploid genome (n=12, C=1 pg) with hundreds of mapped traits and molecular markers (Tanksley, 1993). In addition, tomato is amenable to genetic transformation. Genetic resources such as BAC and YAC libraries are readily available in tomato. The availability of introgression lines containing defined segments of Lycopersicon penneellii chromosome in the L. esculentum background has greatly facilitated gene mapping and map-based cloning in tomato (Eshed and Zamir, 1994). DNA modifying agents, such as EMS, X-rays or fast-neutrons, have been used to generate many useful mutants in tomato. Several references on tomato genetics, gene tagging and mutagenesis can be found in the article by Meissner et al. 1997 and Meissner et al. 2000.

Insertional mutagenesis by T-DNA tagging is a very powerful genetic tool in Arabidopsis, however, it is not very practical in tomato, as transformation procedures are still laborious. Transposon tagging, on the other hand, has been proved to be a promising approach for mutagenesis and gene tagging in tomato The Ac/Ds transposable element family was shown to be active in tomato and patterns of Ac/Ds transposition in this species have been described Many tomato lines containing Ds elements have been produced and mapped in the tomato genome. Availability of these lines makes it possible to take advantage of the preferential insertion of Ac/Ds at nearby sites. This tagging system has been used to isolate several genes, such as Cf9, a locus responsible for Cladosporium resistance and Dwarf, a gene encoding a cytochrome p450 homologue and DCL which controls chloroplast development. Most uses of transposon tagging in tomato, however, have been restricted to target genes that where closely linked to the gene of interest and there are no good tools for high throughput mutagenesis in tomato.

Recently, using the same rationale underlying the Arabidopsis model system, i.e., small size, short life cycle and easy transformation, a tomato model system that greatly enhances the study of tomato genetics and the ability to isolate important genes was developed (Meissner et al. 1997) This system is based on the miniature-dwarf-determinate L. esculentum cultivar, Micro-Tom, originally bred for home gardening purposes. This cultivar can be grown at high density, up to 1350 plants per square meter, yielding mature fruits within 70-90 days from sowing. In addition, genetic stocks consisting of 20,000 EMS mutagenized M2 Micro-Tom plants derived from 9,000 M1 individuals are available (Meissner et al., 1997). A similar size stock of fast-neutron irradiated plants have also been produced in the Micro-Tom background and is also available for mutant screen. In addition, Ac/Ds transposable element enhancer trap and a gene trap systems were introduced into Micro-Tom and found to be active (Meissner et al., 1997). A new generation of gene trap transposons, based on the non-invasive firefly luciferase reporter gene was also developed and 3000 Micro-Tom lines were produced, containing 2-4 transposon copies per lines (Meissner et al. 2000). Considering that 50% of the Ds insertions are into genes, there is a collection of tomato lines with approximately 3000 tagged genes. The infrastructure described above offers a new and unique tool for isolation of genes and mutants in tomato These genetic tools are available for research to isolate mutants and genes that are related to Pi acquisition and utilization in tomato.

Using the Arabidopsis transporters as heterologous probes, two tomato homologues (LePT1 and LePT2) have been identified These homologues share 78-81% amino acid sequence identity with the corresponding Arabidopsis genes (Raghothama, 1999).

Little is known about the genetics of Pi assimilation in tomato. Such knowledge is critical to identification of key molecular determinants involved in this process, and would facilitate further gene isolation or allelic variation identification. So far, only two mutants associated with Pi response have been described: phosphorus-deficiency (pds) on chromosome 6, and miniature phosphorus syndrome (mps) on chromosome 11. Both mutants are retarded in their growth, are chlorotic and purple. The genes involved in these mutations are not known, nor whether they are allelic to LePT1 and/or LePT2.

Thus, the cloning of Pi transporters genes and secreted phosphatases genes from tomato and other plants can be useful for the creation of Pi acquisition-efficient plants.

Expression of Pi transporters and phosphatases will allow reduced application of fertilizers by enhancing Pi acquisition and as such growth of transgenic plants. Since the cloning and characterization of the first high affinity Pi transporter gene from higher plants, a host of Pi transporters have been cloned and characterized from different plants (see references list of Raghothama, 1999). The tremendous interest in Pi transporters reflects the importance and significance of this topic to World Agriculture.

Phosphate as an Environmental Contaminant:

In some cases, the amount of phosphate in soil may be too high for cultivation of certain crops. Such a situation may arise as a result of natural agronomic conditions, or as a result of fertilization protocols employed in the past on a certain plot of land, or as a result of contamination of the aquifer with phosphate fertilizer from another area This problem may be especially severe if the concentration of radioactive phosphate (e.g., ³²P) is higher than normal. Such a situation may require either modification of plants to be cultivated so that they absorb less phosphate from the environment, or removal of the phosphate contamination, for example, by phytoremediation using high Pi insensitive plants and which are further characterized by high Pi uptake.

There is thus a widely recognized need for, and it would be highly advantageous to have, a novel phosphate transporter gene which when expressed in plants can be used to increase root uptake of phosphate from the soil and thereby decrease the requirement for phosphate based fertilizers in crop production. Such a phosphate transporter gene could also be employed to engineer plants for phytoremediation of phosphate contamination. Conversely, reduction of this novel phosphate transporter activity may be desirable in cases where environmental phosphate concentrations are too high.

### SUMMARY OF THE INVENTION

According to one aspect of the present invention there is provided an isolated nucleic acid comprising a genomic, complementary or composite polynucleotide sequence encoding a polypeptide at least 80% homologous to SEQ ID NO:2 as determined using the BestFit software of the Wisconsin sequence analysis package, utilizing the Smith and Waterman algorithm, where gap creation penalty equals 8 and gap extension penalty equals 2, the polypeptide preferably having a phosphate transporter activity.

According to another aspect of the present invention there is provided an isolated nucleic acid comprising a genomic, complementary or composite polynucleotide sequence hybridizable with SEQ ID NO:1 under hybridization conditions of hybridization solution containing 10% dextran sulfate, 1 M NaCl, 1% SDS and 5×10⁶ cpm ³²p labeled probe at 65° C., with a final wash solution of 1×SSC and 0.1% SDS and final wash at 60° C., the polypeptide preferable having a phosphate transporter activity.

According to yet another aspect of the present invention there is provided an isolated nucleic acid comprising a genomic complementary or composite polynucleotide sequence at least 65% identical to SEQ ID NO:1 as determined using the BestFit software of the Wisconsin sequence analysis package, utilizing the Smith and Waterman algorithm, where gap weight equals 50, length weight equals 3, average match equals 10 and average mismatch equals −9, the polynucleotide preferably encodes a polypeptide having a phosphate transporter activity.

According to still another aspect of the present invention there is provided an isolated nucleic acid comprising a polynucleotide sequence as set forth in SEQ ID NO:1.

According to an additional aspect of the present invention there is provided an isolated nucleic acid comprising a polynucleotide sequence encoding a polypeptide as set forth in SEQ ID NO: 2.

According to yet an additional aspect of the present invention there is provided a nucleic acid construct comprising any of the above isolated nucleic acids, the construct is preferably an expression construct and the isolated nucleic acid is oriented therein in a sense or antisense orientation.

According to further features in preferred embodiments of the invention described below, the nucleic acid construct further comprising a promoter for regulating expression of the isolated nucleic acid.

According to still an additional aspect of the present invention there is provided a transformed plant, plant derived tissue or plant cell comprising the nucleic acid construct described herein, preferably as a portion of its genome.

According to a further aspect of the present invention there is provided a pair of oligonucleotides each of at least 17 bases long specifically hybridizable with the isolated nucleic acid of claim 1 in an opposite orientation so as to direct exponential amplification of a portion thereof in a nucleic acid amplification reaction.

According to yet a further aspect of the present invention there is provided a nucleic acid amplification product obtained using the pair of oligonucleotides described above.

According to still a further aspect of the present invention there is provided a recombinant protein comprising a polypeptide sequence at least 75% homologous to SEQ ID NO:2 as determined using the BestFit software of the Wisconsin sequence analysis package, utilizing the Smith and Waterman algorithm, where gap creation penalty equals 8 and gap extension penalty equals 2.

According to another aspect of the present invention there is provided a recombinant protein comprising a polypeptide sequence encoded is by a polynucleotide hybridizable with SEQ ID NO:1 under hybridization conditions of hybridization solution containing 10% dextran sulfate, 1 M NaCl, 1% SDS and 5×10⁶ cpm ³²p labeled probe, at 65° C., with a final wash solution of 1×SSC and 0.1% SDS and final wash at 60° C.

According to yet another aspect of the present invention there is provided a recombinant protein comprising a polypeptide sequence encoded by a polynucleotide at least 65% identical with SEQ ID NO:1 as determined using the BestFit software of the Wisconsin sequence analysis package, utilizing the Smith and Waterman algorithm, where gap weight equals 50, length weight equals 3, average match equals 10 and average mismatch equals −9.

According to still another aspect of the present invention there is provided a recombinant protein comprising a polypeptide sequence as set forth in SEQ ID NO:2.

According to an additional aspect of the present invention there is provided a method of increasing an uptake of phosphate by a plant, the method comprising the step of overexpressing in at least a portion of cells of the plant a phosphate transporter.

According to a further aspect of the present invention there is provided a method for effecting phytoremediation of an area polluted with phosphate, the method comprising the steps of (a) providing a plant overexpressing a phosphate transporter to thereby facilitate uptake and concentration of phosphate within the plant cells; (b) planting the plant in the area polluted with phosphate; (c) following a time period, in which at least a fraction of the phosphate in the area has been accumulated in the plant, harvesting the plant, thereby removing at least the fraction of the phosphate from the area; and optionally (d) repeating steps (b)-(c) until a sufficient amount of the phosphate has been removed from the area.

According to further features in preferred embodiments of the invention described below, the plant is the family Solanaceae, preferably a tomato plant.

According to still further features in the described preferred embodiments the phosphate transporter is encoded by a polynucleotide at least 65% identical with SEQ ID NO:1 as determined using the BestFit software of the Wisconsin sequence analysis package, utilizing the Smith and Waterman algorithm, where gap weight equals 50, length weight equals 3, average match equals 10 and average mismatch equals −9.

According to still further features in the described preferred embodiments the polynucleotide is as set forth in SEQ ID NO:1.

According to still further features in the described preferred embodiments the phosphate transporter is encoded by a polynucleotide hybridizable with SEQ ID NO:1 under hybridization conditions of hybridization solution containing 10% dextran sulfate, 1 M NaCl, 1% SDS and 5×10⁶ cpm ³²p labeled probe, at 65° C., with a final wash solution of 1×SSC and 0.1% SDS and final wash at 60° C.

According to still further features in the described preferred embodiments the phosphate transporter is at least 80% homologous to SEQ ID NO:2 as determined using the BestFit software of the Wisconsin sequence analysis package, utilizing the Smith and Waterman algorithm, where gap creation penalty equals 8 and gap extension penalty equals 2.

According to still further features in the described preferred embodiments at least a portion of the phosphate polluting the area is radioactive.

According to yet an additional aspect of the present invention there is provided an isolated nucleic acid sequence comprising a polynucleotide functional as a plant promoter, wherein the polynucleotide is hybridizable with SEQ ID NO:3 under hybridization conditions of hybridization solution containing 10% dextran sulfate, 1 M NaCl, 1% SDS and 5×10⁶ cpm ³²p labeled probe, at 65° C., with a final wash solution of 1×SSC and 0.1% SDS and final wash at 60° C.

According to further features in preferred embodiments of the invention described below, the polynucleotide is as set forth in SEQ ID NO:3.

According to still an additional aspect of the present invention there is provided an isolated nucleic acid sequence comprising a polynucleotide functional as a plant promoter, wherein the polynucleotide is at least 50% identical with SEQ ID NO:3 as determined using the BestFit software of the Wisconsin sequence analysis package, utilizing the Smith and Waterman algorithm, where gap weight equals 50, length weight equals 3, average match equals 10 and average mismatch equals −9.

According to yet a further aspect of the present invention there is provided a method for generating plants suitable for growth under high phosphate conditions, the method comprising the step of inactivating an expression of an endogenous phosphate transporter gene, the endogenous phosphate transporter gene including a polynucleotide sequence selected from the group consisting of (i) at least 65% identical with SEQ ID NO:1 as determined using the BestFit software of the Wisconsin sequence analysis package, utilizing the Smith and Waterman algorithm, where gap weight equals 50, length weight equals 3, average match equals 10 and average mismatch equals −9; and (ii) hybridizable with SEQ ID NO:1 under hybridization conditions of hybridization solution containing 10% dextran sulfate, 1 M NaCl, 1% SDS and 5×10⁶ cpm ³²p labeled probe at 65° C. with a final wash solution of 1×SSC and 0.1% SDS and final wash at 60° C.

According to further features in preferred embodiments of the invention described below, the step of inactivating an expression of the phosphate transporter gene is effected by a method selected from the group consisting of (i) deleting the endogenous phosphate transporter gene; (ii) mutating and thereby inactivating the endogenous phosphate transporter gene; (iii) transcriptionally inactivating the endogenous phosphate transporter gene; (iv) antisense RNA mediated inactivation of transcripts of the endogenous phosphate transporter gene; (v) translational inactivation of transcripts of the endogenous phosphate transporter gene; and (vi) co-suppression of the endogenous phosphate transporter gene via high copy number transformation.

The present invention successfully addresses the shortcomings of the presently known configurations by providing a phosphate transporter gene which increases the ability of plants to absorb phosphate in soil, either for purposes of phytoremediation or as a means of reducing the need for phosphate based fertilizers. Elimination of the activity of the phosphate transporter can allow cultivation of plants in areas polluted with high concentrations of phosphate.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 depicts the presumed amino acid sequence of the phosphate transporter of the present invention, LePT3 (SEQ ID NO:2) indicating sequence regions homologous with previously described membrane-spanning domains (I to XII, gray boxes) and to phosphorylation sites (black boxes).

FIGS. 2a-b are photographs depicting the phenotype of an LePT3 mutant (FIG. 2a) and wild type (FIG. 2b) tomato plants grown under low (left) and normal (right) phosphate conditions.

FIGS. 3a-b show plant growth of the wild type Micro-Tom plant (WT) and of the LePT3 homozygote mutant (mut) under varying concentration of inorganic Phosphate (Pi) in the irrigation solution. Each point represents the average of 16 plants (four plants per pot and four pots per treatment). Plant growth is expressed as fresh weight (FIG. 3a) or the dry weight (FIG. 3b) of the upper parts of eight-week-old plants (roots were not included).

FIGS. 4a-b show the P uptake of the wild type Micro-Tom plant (WT) and the LePT3 homozygote mutant (mut) under varying concentration of inorganic Phosphate (Pi) in the irrigation solution. Each point represents the average of 16 plants (four plants per pot and four pots per treatment). Pi uptake is expressed as Pi concentration in fresh leaves (FIG. 4a) or total Pi uptake ([Pi]×fresh weight) per plant (FIG. 4b) (roots were not included).

FIGS. 5a-b show the Nitrogen (N) uptake of the wild type Micro-Tom plant (WT) and the LePT3 homozygote mutant (mut) under varying concentration of inorganic Phosphate (Pi) in the irrigation solution. Each point represents the average of 16 plants (four plants per pot and four pots per treatment). N uptake is expressed as N concentration in fresh leaves (FIG. 5a) or total N uptake ([N]×fresh weight) per plant (FIG. 5b) (roots were not included).

FIGS. 6a-b show the Potassium (K) uptake of the wild type Micro-Tom plant (WT) and the LePT3 homozygote mutant (mut) under varying concentration of inorganic Phosphate (Pi) in the irrigation solution. Each point represents the average of 16 plants (four plants per pot and four pots per treatment). K uptake is expressed as K concentration in fresh leaves (FIG. 6a) or total K uptake ([K]×fresh weight) per plant (FIG. 6b) (roots were not included).

### DESCRIPTION OF THE PREFERRED EMBODIMENTS

The present invention is of an isolated polynucleotide encoding a novel phosphate transporter in plants which can be used to modulate phosphate uptake in plants. More particularly, over expressing the isolated polynucleotide encoding the novel phosphate transporter in plants can be used to increase the phosphate uptake of such plants, thus potentially reducing the requirement for phosphate based fertilizers. In addition, over expressing plants can be used for phytoremediation of soils contaminated with phosphate, especially radioactive phosphate isotopes. Finally, inactivation of expression of the endogenous phosphate transporter gene can be used in adapt plants to grow on phosphate rich soils which would otherwise be toxic to such plants.

The principles and operation of the present invention may be better understood with reference to the accompanying descriptions.

Before explaining at least one embodiment of the invention in detail, it is to be understood that the invention is not limited in its application to the details of construction and the arrangement of the components set forth in the following descriptions. The invention is capable of other embodiments or of being practiced or carried out in various ways. Also, it is to be understood that the phraseology and terminology employed herein is for the purpose of description and should not be regarded as limiting.

The uptake system for phosphate and other ions in plants consists of high- and low-affinity components (Epstein, 1976, U Luttge, M G Pittman, eds, Encyclopedia of Plant Physiology, New Series, Vol 2B: Transport in Plants, II, Part B: Tissues and Organs. Springer, Berlin, pp 70-94). Under most natural conditions in which the concentration of available Pi in soil is very low (Barber et al., 1963, J Agric Food Chem 11: 204-207), the transport of Pi by plant cells proceeds through the high-affinity, energy-dependent proton/phosphate symport mechanism. At the molecular level, the main protein component of this system, the phosphate transporter, has been recently characterized from Arabidopsis (Muchhal et al., 1996, Proc Natl Acad Sci USA 93: 10519-10523; Smith et al., 1997, Plant J 11: 83-92), potato (Leggewie et al., 1997, Plant Cell 9: 381-392), and C. roseus (Kai et at., 1997, Soil Sci Plant Nutr 43: 227-235). These proteins show significant structural similarity with known high-affinity phosphate transporters and were able to complement yeast mutants defective in high-affinity phosphate uptake activity ((Muchhal et al., 1996, Proc Natl Acad Sci USA 93: 10519-10523; Kai et al., 1997, Soil Sci Plant Nutr 43: 227-235; Leggewie et al., 1997, Plant Cell 9: 38l-392). Furthermore, overexpression of AtPT1 (PHT1) in tobacco cell cultures enhanced cell growth and Pi uptake under phosphate-limited conditions (Mitsutkawa et al., 1997, Proc Natl Acad Sci USA 94: 7098-7102).

The deduced amino acid sequences of tomato phosphate transporters LePT1 and LePT2, show a high degree of sequence similarity to other plant phosphate transporters. LePT1 and LePT2 are greater than 95% identical at the amino acid sequence level to STPT1 and STPT2 from potato, respectively. This degree of similarity at the primary sequence level among the members of the Solanaceae family is interesting, considering the fact that, despite similar functionality, the overall sequence similarity among phosphate transporters from plants and fungi is not more than 42% (Muchhal et al., 1996, ibid).

Results from expression studies show that both LePT1 and LePT2 transcripts accumulate primarily in roots, and their expression is highly induced under Pi-deficient conditions. Roots are the organs involved in nutrient acquisition, and the expression pattern of these two genes in roots correlates well with their function. A small amount of LePT1 message was also detectable in the leaves, stem, and petioles of tomato plants starved of Pi, suggesting a global role for this transporter in plants. The differential expression of LePT1 and LePT2 in roots and leaves was similar to that of potato phosphate transporters described by Leggewie et al. (1997, ibid). The induction of LePT1 and LePT2 in response to Pi starvation correlates well with published reports of increased phosphate uptake rate of roots and cell cultures subjected to Pi deprivation (Clarkson and Scattergood, 1982, J Exp Bot 33: 865-875; Drew and Saker, 1984, Planta 160: 500-507; Katz et al., 1986, Physiol Plant 67: 23-28). This enhanced Pi absorption following Pi starvation has been proposed to be associated with a larger capacity for Pi transport, possibly by the formation of additional carriers for Pi (Anghinoni and Barber, 1980, Agron J 72: 685-688; Lefebvre and Glass, 1982, Physiol Plant 54: 199-206; Drew et al., 1984, Planta 160: 490-499; Furihata et al., 1992, Plant Cell Physiol 33: 1151-1157; Shimogawara and Usuda, 1995, Plant Cell Physiol 36: 341-351).

Increased transcription of phosphate-transporter genes in Pi-limiting conditions has been well documented in several microorganisms (Torriani-Gorini et al., 1994, Phosphate in Microorganisms: Cellular and Molecular Biology, American Society of Microbiology, Washington, D.C.).

In Saccharomyces cerevisiae, which, like plants, has both high- and low-affinity Pi uptake systems, transcription of the high-affinity phosphate transporter (PHO84) is controlled by the availability of Pi in the medium through the action of several positive and negative regulators constituting the pho regulon (Youshida et al., 1987, Phosphate Metabolism and Circular Regulation in Microorganisms. American Society for Microbiology, Washington, D.C., pp 49-55; Johnston and Carlson, 1992, The Molecular and Cellular Biology of the Yeast Saccharomyces: Gene Expression, Cold Spring Harbor Laboratory Press, Cold Spring Harbor, N.Y., pp 193-281). By analogy, a similar feedback system for controlling the activity of Pi transporters in response to external Pi availability could be postulated for plants, since their expression appears to be linked to the availability of Pi. However, the situation in plants is much more complex, considering that Pi is a highly mobile element within plants, and its uptake has to be coordinated with growth requirements of tissues far away from the point of uptake.

Plants have developed several adaptive mechanisms to cope with limiting concentrations of Pi available in soils, most of which focus on increasing the availability of external Pi and efficiency of Pi uptake and utilization inside. However, in the constantly fluctuating environment surrounding roots, the most important response is fine tuning of Pi uptake rate relative to the availability of Pi. Transcriptional regulation of Pi-transporter gene expression, regulated directly or indirectly by internal Pi, appears to be a very important part of this control mechanism in roots.

Such prior art studies which were conducted in efforts to uncover the regulation of phosphate transporter expression in plants monitored the expression patterns of two phosphate transporter genes (LePT1 and LePT2 in tomato).

However, as uncovered by studies performed as part of the present invention, phosphate uptake in tomato plants and probably other Solanaceae relies mainly on a third type of phosphate transporter which is designated herein as LePT3 and which shares only a partial and remote sequence homology with either LePT1 or LePT2 (Table 1, Example 1).

As is further described hereinbelow in Examples 1-3 of the Examples section, the phosphate transporter gene of the present invention is essential for maintenance of the wild type phenotype in tomato plants under low phosphate conditions. This fact indicates that regulation of the LePT3 gene is the key to engineering plants for growth under both conditions in which phosphate insufficiency in the soil is a problem and under conditions in which phosphate excess is a problem.

Thus, according to one aspect of the present invention there is provided an isolated nucleic acid including a genomic, complementary or composite polynucleotide sequence, which isolated nucleic acid sequence encodes a polypeptide having an essential phosphate transporter activity.

As used herein the phrase “essential phosphate transporter activity” denotes a phosphate transporter activity which, when absent, leads to a very severe phosphate deficiency syndrome under low phosphate conditions and to a milder effect and growth retardation under otherwise optimal phosphate conditions, as, for example, is further described in Examples 1-3 of the Examples section.

The polypeptide encoded by the isolated nucleic acid according to a preferred embodiment of this aspect of the present invention is at least 70%, at least 75%, at least 80%, at least 85%, at least 90%, at least 95%, and/or 98-100% homologous (similar+identical amino acids) to SEQ ID NO:2 (LePT3) as determined using the BestFit software of the Wisconsin sequence analysis package, utilizing the Smith and Waterman algorithm, where gap creation penalty equals 8 and gap extension penalty equals 2.

As used herein in the specification and in the claims section that follows, the phrase “complementary polynucleotide sequence” includes sequences which originally result from reverse transcription of messenger RNA using a reverse transcriptase or any other RNA dependent DNA polymerase. Such sequences can be subsequently amplified in vivo or in vitro using a DNA dependent DNA polymerase.

As used herein in the specification and in the claims section that follows, the phrase “genomic polynucleotide sequence” includes sequences which originally derive from a chromosome and reflect a contiguous portion of a chromosome.

As used herein in the specification and in the claims section that follows, the phrase “composite polynucleotide sequence” includes sequences which are at least partially complementary and at least partially genomic.

As used herein the terms “homology” or “homologous” refer to the resemblance between compared polypeptide sequences as determined from the identity (match) and similarity (amino acids of the same group) between amino acids which comprise these polypeptide sequences.

Preferably, the isolated nucleic acid according to this aspect of the present invention is hybridizable with SEQ ID NO:1 under moderate to stringent hybridization conditions.

Hybridization under moderate hybridization conditions is effected by a hybridization solution containing 10% dextrane sulfate, 1 M NaCl, 1% SDS and 5×10⁶ cpm ³²p labeled probe, at 65° C. with a final wash solution of 1×SSC and 0.1% SDS and final wash at 55 to 65, preferably 60° C. whereas, hybridization under stringent hybridization conditions is effected by a hybridization solution containing 10% dextrane sulfate, 1 M NaCl, 1% SDS and 5×10⁶ cpm ³²p labeled probe, at 65° C, with a final wash solution of 0.1×SSC and 0.1% SDS and final wash at 55 to 65, preferably 60° C.

Alternatively or additionally, the isolated nucleic acid according to this aspect of the present invention is at least 60%, at least 65%, at least 70%, at least 75%, at least 80%, at least 85%, at least 90%, at least 95%, preferably 98-100% identical with SEQ ID NO:1 as determined using the BestFit software of the Wisconsin sequence analysis package, utilizing the Smith and Waterman algorithm, where gap weight equals 50, length weight equals 3, average match equals 10 and average mismatch equals −9.

Sequences of the isolated nucleic acid according to this aspect of the present invention encode the LePT3 phosphate transporter protein (SEQ ID NO:2). As further detailed in Examples 1-3 of the Examples section, LePT3 mutant tomato plants display a phosphate deficient phenotype which means that LePT1 and LePT2 are not able to compensate for its absence and that LePT3 plays a major role in Pi uptake. This feature was not shown for other Pi transporters, as under Pi starvation, LePT1 and LePT2 fail to compensate for the absence of LePT3 although are known to be transcriptionally activated under such starvation conditions. As such, it is said to be “essential”.

According to a preferred embodiment of the present invention the isolated nucleic acid is as set forth in SEQ ID NO:1.

According to a preferred embodiment of the present invention the polypeptide encoded by the isolated nucleic acid is as set forth in SEQ ID NO:2.

According to another aspect of the present invention there is provided an isolated nucleic acid functional as a plant promoter The isolated nucleic acid according to this aspect of the present invention is at least 50%, at least 55%, at least 60%, at least 65%, at least 70%, at least 75%, at least 80%, at least 85%, at least 90%, at least 95%, 95-100% identical with SEQ ID NO:3 as determined using the BestFit software of the Wisconsin sequence analysis package, utilizing the Smith and Waterman algorithm, where gap weight equals 50, length weight equals 3, average match equals 10 and average mismatch equals −9.

According to another preferred embodiment of this aspect of the present invention, the isolated nucleic acid is as set forth in SEQ ID NO:3 or any functional portion or derivatives and/or modificants (e.g., mutants) thereof. No homologous sequences could be identified for SEQ ID NO:3 in the GeneBank. SEQ ID NO:3 includes a classical TATA promoter motif.

The nucleic acid sequences of the present invention may be amplified by known nucleic acid amplification protocols, such as, but not limited to, the polymerase chain reaction (PCR). To this end, there is provided a pair of oligonucleotides each of at least 17, preferably at least 18, more preferably between 19 and 25, still preferably, at least 26, say, 27-50 bases in length, which are specifically hybridizable with the isolated nucleic acid described herein in an opposite orientation, so as to direct exponential amplification of a portion thereof in a nucleic acid amplification reaction, thereby obtaining a nucleic acid amplification product. The melting temperature (Tm) of the oligonucleotides of the pair of nucleotides is preferably selected similar so as to enable hybridization thereof to target sequences under similar conditions. Such melting temperature can be estimated in advance using an appropriate software, such as, but not limited to the OLIGO software. Sequence mutated or modified oligonucleotides can be used for various purposes, such as, but not limited to, the introduction of mutation(s) or other sequence alterations in the resulting sequence.

Another aspect of the present invention relates to a recombinant protein. According to one embodiment, the recombinant protein comprises a polypeptide sequence at least 75% homologous to SEQ ID NO:2 as determined using the BestFit software of the Wisconsin sequence analysis package, utilizing the Smith and Waterman algorithm, where gap creation penalty equals 8 and gap extension penalty equals 2.

According to another embodiment, the recombinant protein comprises a polypeptide sequence encoded by a polynucleotide hybridizable with SEQ ID NO:1 under hybridization conditions of hybridization solution containing 10% dextran sulfate, 1 M NaCl, 1% SDS and 5×10⁶ cpm ³²p labeled probe, at 65° C., with a final wash solution of 1×SSC and 0.1% SDS and final wash at 60° C.

According to one embodiment, the recombinant protein comprises a polypeptide sequence encoded by a polynucleotide at least 65% identical with SEQ ID NO:1 as determined using the BestFit software of the Wisconsin sequence analysis package, utilizing the Smith and Waterman algorithm, where gap weight equals 50, length weight equals 3, average match equals 10 and average mismatch equals −9.

Preferably, the recombinant protein comprises a polypeptide sequence as set forth in SEQ ID NO:2.

The recombinant protein of the present invention is a membrane embedded protein and as such may be isolated integrated within or reconstituted into liposomes.

Scientific evidence presented by studies performed while reducing the present invention to practice have shown that the phosphate transporter of the present invention is essential for phosphate uptake in tomato plants, and as such it is responsible for a major portion of the phosphate uptake.

Thus, overexpression of the transporter will substantially increase phosphate uptake in plants. Such overexpression can be effected, for example, by transforming plants with the nucleic acid of the present invention as is further detailed below. Alternatively, it can be effected by introducing upregulating cis acting control elements upstream to the endogenous gene encoding the transported in a plant, using, for example, gene knock-in techniques which are further detailed hereinunder. As such, the present invention enables increasing the uptake of phosphate by a plant such as, for example, a plant of the Solanaceae family.

As used herein the term “overexpression” refers to expressing a protein to a level higher than that found naturally in the plant. Over expression can be effected by transforming the plant with a high copy number of the nucleic acid, by using a strong promoter and/or transcriptional enhancers to generate a higher transcript level, or by utilizing cis acting sequences which stabilize the resultant transcript and as such decrease the degradation or “turn-over” of such a transcript.

As used herein the term “transformed plant” refers to a plant which includes an exogenous nucleic acid sequence either stabely integrated into the plant genome, and as such inherited by a progeny of such a plant (i.e., transgenic plant), or present in the nucleus, other organelle or cytosol in a transient manner, and as such not inherited by a progeny of such a plant.

For effecting plant transformation, the isolated nucleic acid of the present invention is preferably included within a nucleic acid construct which serves to facilitates the introduction of the isolated nucleic acid in plant cells or tissues and the expression of the protein in the plant.

The nucleic acid construct according to the present invention is utilized to express either in a stable or transient manner the isolated nucleic acid within a plant, plant derived tissues, or plant cells either possessing a cell wall or not (protoplasts).

Thus, according to a preferred embodiment of the present invention, the nucleic acid construct further includes a promoter for regulating expression of the isolated nucleic acid in a sense or antisense orientation.

Numerous plant functional expression promoters and enhancers which can be either tissue specific: developmentally specific, constitutive or induced and which can be utilized by the construct of the present invention, some examples are provided hereinunder.

As used herein in the specification and in the claims section that follows the phrase “plant promoter” or “promoter” includes a promoter which can direct gene expression in plant cells (including DNA containing organelles) Such a promoter can be derived from a plant, bacterial, viral, fungal or animal origin Such a promoter can be constitutive, i.e. capable of directing high level of gene expression in a plurality of plant tissues, tissue specific, i.e., capable of directing gene expression in a particular plant tissue or tissues, inducible i.e., capable of directing gene expression under a stimulus, or chimeric, i.e. formed of portions of at least two different promoters.

Thus, the plant promoter employed can be a constitutive promoter, a tissue specific promoter, an inducible promoter or a chimeric promoter.

Examples of constitutive plant promoters include, without being limited to, CaMV35S and CaMV19S promoters, FMV34S promoter, sugarcane bacilliform badnavirus promoter, CsVMV promoter, Arabidopsis ACT2/ACT8 actin promoter, Arabidopsis ubiquitin UBQ1 promoter, barley leaf thionin BTH6 promoter, and rice actin promoter.

Examples of tissue specific promoters include, without being limited to, bean phaseolin storage protein promoter, DLEC promoter, PHSβ promoter, zein storage protein promoter, conglutin gamma promoter from soybean, AT2S1 gene promoter, ACT11 actin promoter from Arabidopsis, napA promoter from Brassica napus and potato patatin gene promoter.

The inducible promoter is a promoter induced by a specific stimuli such as stress conditions comprising, for example, light, temperature, chemicals, drought, high salinity, osmotic shock, oxidant conditions or in case of pathogenicity and include, without being limited to, the light-inducible promoter derived from the pea rbcS gene, the promoter from the alfalfa rbcS gene, the promoters DRE, MYC and MYB active in drought; the promoters INT, INPS, prxEa, Ha hsp17-7G4 and RD21 active in high salinity and osmotic stress- and the promoters hsr203J and str246C active in pathogenic stress.

Since phosphate transporter activity is most essential in the roots, the promoter of the vector construct according to the present invention is preferably a root specific promoter, such as, but not limited to the promoters of the ARSKS1 (Hwang and Goodman 1995) or the MsPR2 (Bastola et al., 1998) genes.

In addition the promoter encoded by SEQ ID NO:3 can also be utilized to express the protein, especially if high copy number is of choice. It is believed, yet by all means not binding, that this promoter is root specific and optionally inducible.

The construct according to the present invention preferably further includes an appropriate selectable marker, such as, for example, an antibiotic resistance gene In a more preferred embodiment according to the present invention the construct further includes an origin of replication.

The construct according to the present invention can be a shuttle vector, which can propagate both in E. coli (wherein the construct comprises an appropriate selectable marker and origin of replication) and be compatible for propagation in cells, or integration in the genome, of a plant. The construct according to this aspect of the present invention can be, for example, a plasmid, a bacmid, a phagemid, a cosmid, a phage, a virus or an artificial chromosome.

As already mentioned hereinabove, the construct is introduced into the plant in order to express the protein encoded by the isolated nucleic acid of the present invention therein.

There are various methods of introducing nucleic acid constructs into both monocotyledonous and dicotyledenous plants (Potrykus, I., Annu. Rev. Plant. Physiol., Plant. Mol. Biol (1991) 42:205-225; Shimamoto et al., Nature (1989) 338:274-276). Such methods rely on either stable integration of the nucleic acid construct or a portion thereof into the genome of the plant, or on transient expression of the nucleic acid construct in which case these sequences are not inherited by a progeny of the plant.

There are two principle methods of effecting stable genomic integration of exogenous nucleic acid sequences such as those included within the nucleic acid construct of the present invention into plant genomes:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; or by the direct incubation of DNA with germinating pollen, DeWet et al. in Experimental Manipulation of Ovule Tissue, eds. Chapman, G. P. and Mantell, S. H. and Daniels, W. Longman, London, (1985) p 197-209; and Ohta, Proc. Natl. Acad. Sci. USA (1986) 83:715-719.

The Agrobacterium system includes the use of plasmid vectors that contain defined DNA segments that integrate into the plant genomic DNA, Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledenous plants.

There are various methods of direct DNA transfer into plant cells. In electroporation, protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals, tungsten particles or gold particles, and the microprojectiles are physically accelerated into cells or plant tissues.

Following transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Transient expression methods which can be utilized for transiently expressing the isolated nucleic acid included within the nucleic acid construct of the present invention include, but are not limited to, microinjection and bombardment as described above but under conditions which favor transient expression and viral mediated expression wherein a packaged or unpackaged recombinant virus vector including the nucleic acid construct is utilized to infect plant tissues or cells such that a propargating recombinant virus established therein expresses the non-viral nucleic acid sequence.

Viruses that have been shown to be useful for the transformation of plant hosts include CaV, TMV and BV. Transformation of plants using plant viruses is described in U.S. Pat. No. 4,855,237 (BGV), EP-A 67,553 (TMV), Japanese Published Application No.63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, the constructions can be made to the virus itself Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA sill produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of the present invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment a recombinant plant viral nucleic acid is provided as in the first embodiment except that the native coal protein coding sequence is placed adjacent one of the non-native coal protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that said sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In another embodiment of the present invention overexpression is achieved via enhancing the expression of the endogenous protein. To this end, a cis acting expression upregulating sequence, such as any one of the promoters listed above or an enhancer is recombinantly introduced preferably upstream to, or Into an intron of the endoenous LePT3, so as to enhance transcription therefrom, using recombinant knock-in constructs and methods.

Gene knock-in constructs including sequences homologous to the region of integration as well as non-homologous sequences to be inserted into the genome thereat. The knock-in construct according to this aspect of the present invention further include a positive and a negative selection markers and may therefore be employed for selecting for homologous to recombination events. One ordinarily skilled in the art can readily design a knock-in construct including both positive and negative selection genes for efficiently selecting transformed plant cells that underwent a homologous recombination event with the construct. Such cells can then be grown into full plants. Standard methods known in the art can be used for implementing knock-in procedure, such methods are set forth in, for example, U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson, Methods in Enzymology, 194:251-270, 1991; Capecchi, Science 244:1288-1292, 1989; Davies et al., Nucleic Acids Research, 20 (11) 2693-2698, 1992; Dickinson et al., Human Molecular Genetics, 2(8):1299-1302, 1993; Duff and Lincoln. “Insertion of a pathogenic mutation into a yeast artificial chromosome containing the human APP gene and expression in ES cells”, Research Advances in Alzheimer's Disease and Related Disorders, 1995; Huxley et al., Genomics, 9:742-750 1991; Jakobovits et al., Nature, 362:255-261 1993; Lamb et al., Nature Genetics, 5: 22-29, 1993; Pearson and Choi, Proc. Natl. Acad. Sci. USA, 1993, 90:10578-82; Rothstein, Methods in Enzymology, 194:281-301, 1991; Schedl et al., Nature, 362: 258-261, 1993. Strauss et al., Science, 259:1904-1907, 1993, WO 94/23049, WO93/14200. WO 94/06908 and WO 94/28123 also provide information.

Plants over expressing the transporter of the present invention and which are generated as described above, greatly reduce the need for phosphate based fertilizers since uptake efficiency in such plants is greatly enhanced.

In addition to reducing phosphate based fertilizer requirements, such plants can also be utilized for phytoremediation of an area polluted with phosphate Such plants are preferably, yet not obligatorily inherently insensitive to high phosphate conditions.

Thus, plants overexpressing the transporter of the present invention can be planted in an area polluted with phosphate, and following a time period, in which at least a fraction of the phosphate in the area has been accumulated in the plants, the plants are harvested, thereby removing at least the fraction of the phosphate from the area. Such a procedure can be repeated any number of times until the area polluted is deemed free of polluting phosphate.

Plants overexpressing the transporter of the present invention and which are generated according to the teachings of the present invention are especially useful in the phytoremediation of areas highly contaminated with ³²P or areas in which toxic levels of phosphate exist.

Since phosphate is readily assimilated in the plant, plants can accumulate high concentrations of phosphate and as such serve as an excellent phosphate sink in phosphate polluted areas.

In some cases, the amount of phosphate in soil may be too high for cultivation of certain crops. Such a situation may arise as a result of natural agronomic conditions, or as a result of fertilization protocols employed in the past on a certain plot of land, or as a result of contamination of the aquifer with phosphate fertilizer from another area.

Thus according to another aspect of the present invention a plant suitable for growth under such conditions can be generated by inactivating the expression of the plant's endogenous phosphate transporter, such as, but not limited to. LePT3, LePT2 and/or LePT1.

To inactivate the expression of the phosphate transporter one or more of the following approaches can be utilized: (i) deleting the endogenous phosphate transporter gene; (ii) mutating and thereby inactivating the endogenous phosphate transporter gene; (iii) transcriptionally inactivating the endogenous phosphate transporter gene; (iv) antisense RNA mediated inactivation of transcripts of the endogenous phosphate transporter gene; (v) translational inactivation of transcripts of the endogenous phosphate transporter gene; and (vi) co-suppression of the endogenous phosphate transporter gene via high copy number transformation. These methods are well known in the art. Nevertheless, some description is provided hereinafter.

Thus, for example, gene knock-in or gene knock-out constructs including sequences homologous with the endogenous phosphate tansporter gene can be generated as is further detailed hereinabove and be used to insert an ancillary sequence into the coding sequence of a transporter gene, to thereby inactivate this gene.

At the transcription level, expressing antisense or sense oligonucleotides that bind to the genomic DNA by strand displacement or the formation of a triple helix, may prevent transcription. At the transcript level, expression of antisense oligonucleotides that bind target mRNA molecules lead to the enzymatic cleavage of the hybrid by intracellular RNase H or prevention of translation thereof into a protein. In this case, by hybridizing to the targeted mRNA, the oligonucleotides provide a duplex hybrid recognized and destroyed by the RNase H enzyme or which prevents binding to ribosomes. In addition the use of ribozyme sequences linked to antisense oligonucleotides can also facilitate target sequence cleavage by the ribozyme. Alternatively, such hybrid formation may lead to interference with correct RNA splicing into messenger RNA. As a result, in all cases, the number of the target mRNA intact transcripts ready for translation is reduced or eliminated At the translation level, antisense oligonucleotides or analogs that bind target mRNA molecules prevent, by steric hindrance, binding of essential translation factors (ribosomes), to the target mRNA, a phenomenon known in the art as hybridization arrest, disabling the translation of such mRNAs.

Thus, the present invention provides a novel phosphate transporter gene which can be used to generate plants which can be cultivated on phosphate deficient soil and for phytoremediation of phosphate polluted soils. In addition, the present invention further provides a method for growing plants on phosphate rich soils which may be unsuitable for the cultivation of certain crops.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons Baltimore, Md. (1989); Perbal, “A Practical Guide to Molecular Cloning”, John Wiley & Sons, New York (1988); Watson et al. “Recombinant DNA”. Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Colloan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034.074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials ad Experimental Methods

The following materials and methods are provided in conjunction with the specific examples hereinbelow.

Plants:

The plant material used to isolate the LePT3 gene consists of a population of 3000 tomato lines, each of which contains 2-3 copies of a Ds transposon insertion. See, WO/99/12411, which is incorporated herein by reference.

Isolation of Ds-Flanking Regions:

Genomic DNA was extracted from 20 pools of 5 Ds-insertion lines. Each pool was digested with one of the three restriction enzymes: SpeI, XbaI, and Asp718. These enzymes do not digest sequences within the Ds element, therefore resultant restriction fragments contain sequences of Ds and of its flanking region. The DNA was diluted and the ends of the restriction fragments were joined by ligation with T4-DNA ligase. The ligation products were then used as a template for inverse-PCR with primers corresponding to Ac/Ds termini as described (Gorbunova and Levy 1997). The PCR products were run on a gel, and discrete fragments were purified with GeneClean as described in the Manufacturer's instructions and directly sequenced using a BigDye Terminator Cycle Sequencing Kit from ABI and Applied Biosystems DNA Sequencer.

### EXAMPLE 1

### Discovery of a New Member of the Pi Transporter's Family (LePT3)

The sequencing of transposon-flanking regions (TFRs) obtained by inverse-PCR amplification, yielded several fragments that were sequenced. One of the 3 Kb DNA fragments obtained by this method showed homology at the amino-acid level to the tomato high affinity Pi transporters encoded by the LePT1 and LePT2 genes. Some homology with other Pi transporters from plants and fungi was also observed. This new member of the Pi transporter family in tomato is therefore referred to herein as LePT3. The mutant allele contains a Ds insertion in the middle of the LePT3 gene. This insertion caused a 540 bp deletion upon integration, possibly leading to a null mutation. In a first step, only a partial sequence of the gene was obtained. Further walking by Inverse-PCR enabled isolation of the full genomic sequence of the gene (SEQ ID NO:1), as well as some upstream regions that contain the gene's promoter (SEQ ID NO:3). The genomic DNA sequence of LePT3 (SEQ ID NO:1) was co-aligned with DNA sequences from all GeneBank database. Table 1 below summarizes the alignment results for the best hits. None of the homologous sequences displayed identity greater than 62.2% to the sequence of LePT3 as shown in SEQ ID NO:1.

TABLE 1
Best hits from the alignment between LePT3
genomic DNA and DNA sequences ftwn GeneBank

| Accession No. | Description | E-value | % Identity | Gaps |
|---|---|---|---|---|
| AF156695.1 | PT1 inorganic phosphate transporter | 1e−35 | 62.2 | 55 |
| | S.tuberosum | | | |
| X98890 | STPT1 S.tuberosum mRNA for inorganic | 1e−35 | 60.6 | 6 |
| | phosphate transporter | | | |
| Y16125 | Lycopersicon esculentum mRNA for putative | 3e−33 | 59 | 6 |
| | phosphate transporter | | | |
| AF022873 | LePT1 Lycopersicon esculentum inorganic | 3e−33 | 594 | 6 |
| | phosphate transporter | | | |
| AB020061.1 | Nicotiana tabacum mRNA for Phosphate | 4e−23 | 609 | |
| | transporter | | | |

As is shown in Table 2, the presumed amino acid sequence (SEQ ID NO:2) of LePT3 was compared to the translated amino acid sequences of DNAs of other sequences in the GeneBank database, None of the translated sequences displayed identity greater than 64% or homology greater than 78% to the LePT3 translated sequence.

TABLE 2
Best hits from the aliginment between LePT3
protein and protein sequences from GeneRank

| | | | % identity |
|---|---|---|---|
| Accession* | Description | E-Value | (% homology) |
| Y14214 | LePT1 phosphate transporter L. esculentum | e−175 | 64(78) |
| AF022873 | LePT1 inorganic phosphate transporter L. esculentum | e−175 | 61 (75) |
| A8000094 | PHT3 inorganic phosphate transporter A. thaliana | e−175 | 60 (73) |
| U97546 | AtPT4 putative proton/phosphaic corransporter A. thaliana | e−174 | 60(73) |
| Y07682 | APT2 phosphate transporterA. thaliana | e−174 | 59(72) |
| U62330 | AIPT1 phosphate transporter A. thaliana | e−174 | 59 (72) |
| AB000094 | PHT2 inorganic phosphate transporter A. thaliana | e−174 | 60(73) |
| AF000355 | MtPT2 phosphate transporter M. truncatula | e−173 | 60(74) |
| AF022874 | LePT2 inorganic phosphate transporter L. esculentum | e−173 | 60 (74) |
| X98890 | StPT1 inorganic phosphate transporter S. tuberosum | e−173 | 60 (74) |
| Y16125 | Putative phosphate transporter L. esculentum | e−173 | 60(74) |
| AF156695 | PT1 inorganic phosphate transporter S. tuberosum | e−173 | 60 (75) |
| Y07681 | APT1 phosphate transporter A. thaliana | e−173 | 60 (73) |
| AB004809 | PIT1 phosphate transporter Catharanthus roseus | e−173 | 59 (73) |
| AB020061 | Phospate transpoter N. tabacum | e−173 | 60 (74) |
| AF000354 | MtPT1 phosphate transporter M. truncatula | e−172 | 59 (73) |
| AC003033 | Putative phosphate transporter A. thaliana | e−171 | 59 (74) |
| U62331 | ALPT2 phosphate transporter A. thaliana | e−171 | 57 (72) |
| X98891 | StPT2 inorganic phosphate transporter 2 S. tuberosum | e−165 | 59 (73) |
| AB005746 | Inorganic phosphate transporter A. thaliana | e−165 | 56 (74) |
| AF110180 | PT1 high-affinity phosphate transport T. aestivum | e−132 | 60 (72) |
| AC012394 | FISM4.7 putative phosphate transporter A. thaliana | e−123 | 47 (66) |
| AF128396 | T3H13.9 similar to phosphate transporters A. thaliana | 1e−84 | 58 (73) |
| AB011417 | Phosphate permease G. zea | e3e−84 | 38(57) |
| pir S67491 | Phosphate transporter G. versforme | 5e−84 | 38 (55) |
| PHOS-4 | PHOS4 inorganic phosphate transporter S. cerevisiae | e−70 | 35 (54) |
| L36127 | Phosphate permease N. crassa | e−56 | 32 (48) |

*GeneBank Accession No. ** % homology = identical + similar amino acids.

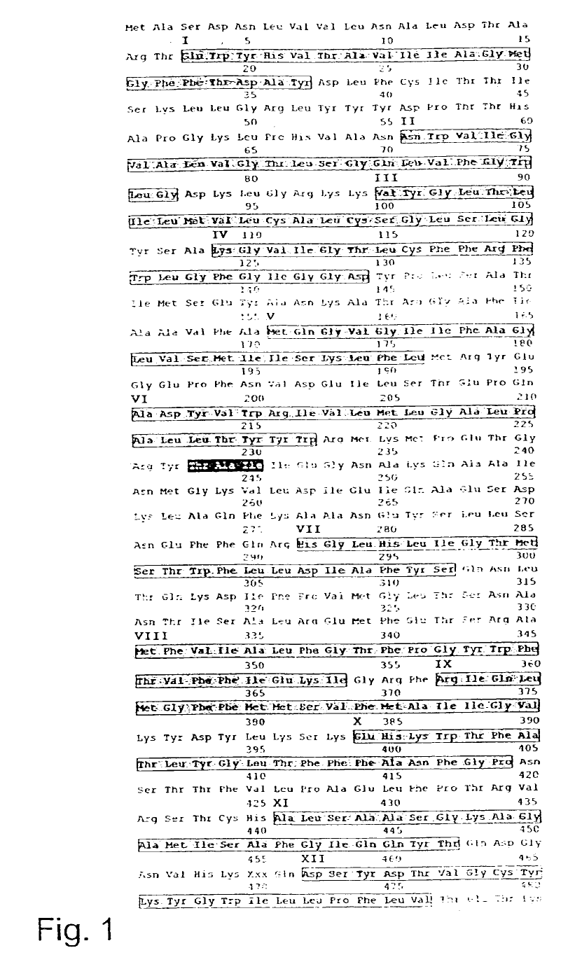

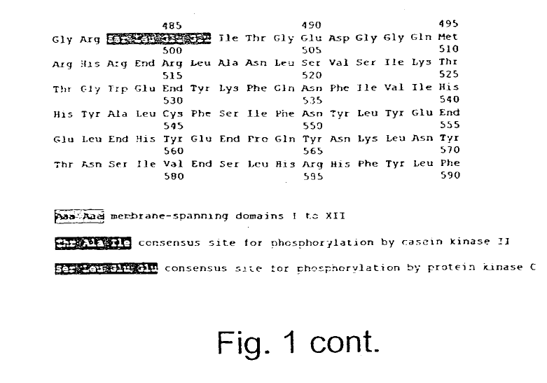

In addition, sequence analysis of LePT3 revealed 12 transmembrane domains and putative casein kinase and protein kinase C phosphorylation sites (FIG. 1).

### EXAMPLE 2

### Functional Analysis of the LePT3 Mutant

Despite some homology to several other plant Pi transporters, it was not clear whether LePT3 plays an important role in Pi acquisition. Moreover, since two additional transporters (LePT1 and LePT2) have been previously characterized, the function of LePT3 in plants may be redundant. Thus, functional analysis of an LePT3 mutant was effected in order to determine the function of this gene.

Seeds from homozygous LePT3 mutant plants were obtained and their growth rate under different concentrations of Pi was determined in an experiment including four replicas per treatment and four plants in each replica. Plants were grown in 10 liter-pots, in perlite, and Pi was provided in the irrigation at varying concentrations while the other major nutrients, namely the Nitrates (N) and potassium (K) were fixed.

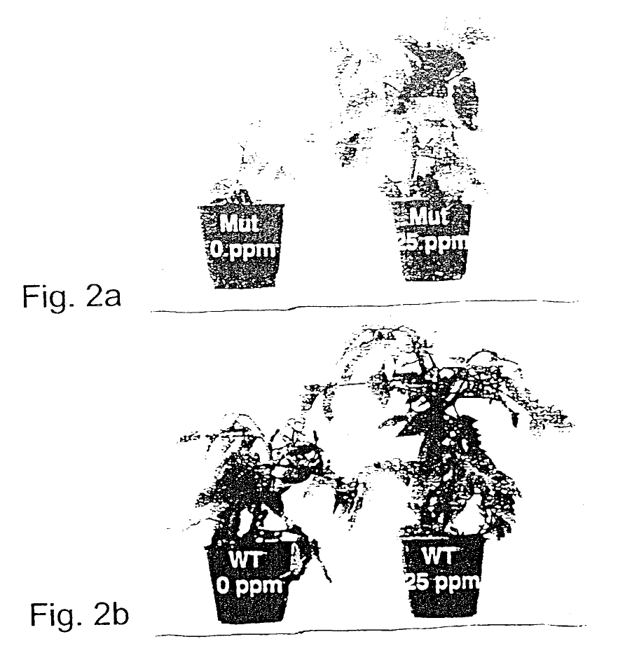

In the absence of Pi (0 mg/l) strong Pi deficiency symptoms were observed in the mutant plant but not in the WT plant. Plants of the LePT3 mutant line displayed inhibited growth patterns (FIG. 2) with yellowish leaves including purple (anthocyanin).

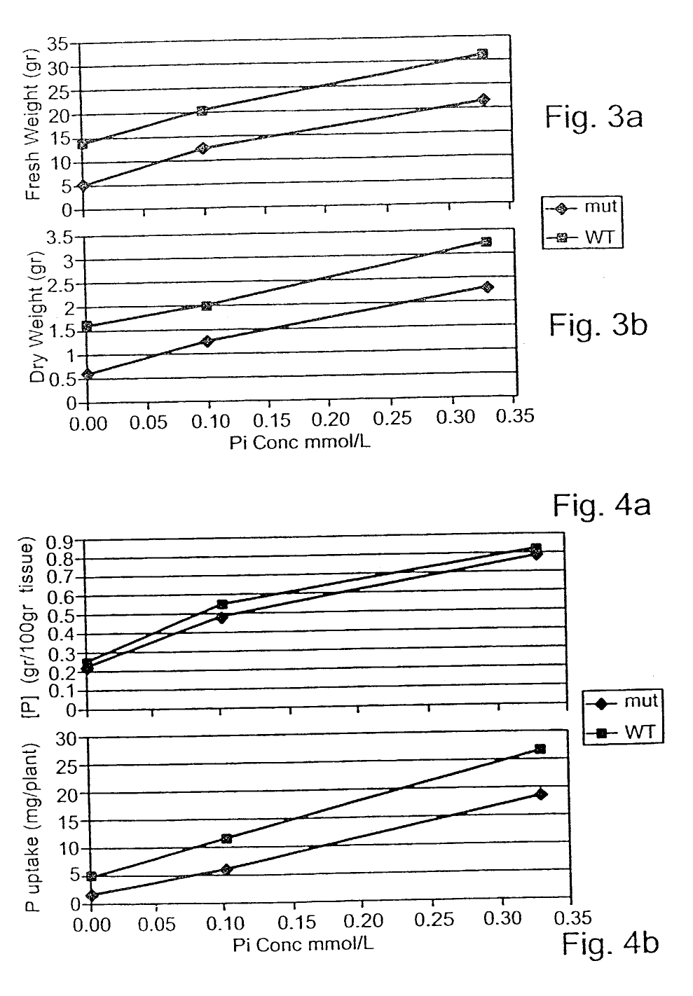

At higher levels of Pi, the difference in biomass between mutant and wild type was not visible. However, weight measurement of all the replica revealed significant differences in growth as expressed by fresh or dry weight (FIG. 3).

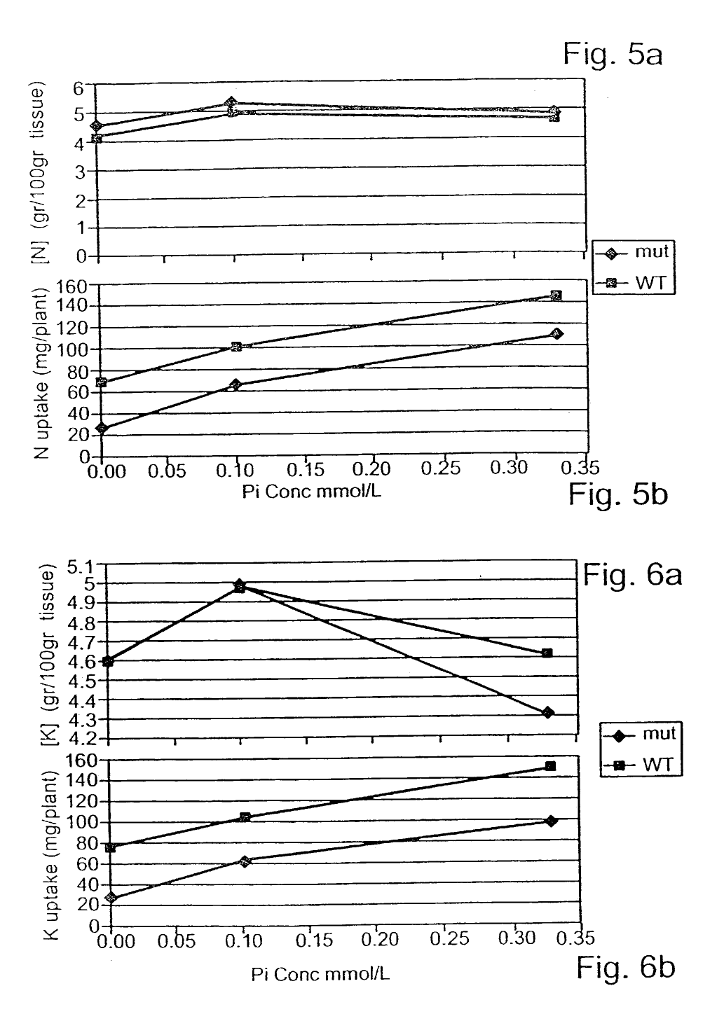

In addition, mineral uptake was determined in the plants crown as described above. Interestingly, there was no difference between the mutant and wild tape in the concentration of Phosphate in the plant (FIG. 4a). However, the total P uptake was greatly reduced in the mutant because of the inhibition in plant growth (FIG. 4b). Similarly, in the mutant, there was a reduction of uptake of the other essential plant nutrients (N and K) as is shown in FIGS. 5 and 6, probably because of the growth inhibition.

This experiment clearly demonstrates that LePT3 plays an important role in plant growth through Pi acquisition, and indirectly through the acquisition of other major nutrients. It is also clear that LePT1 and LePT2 cannot compensate for the absence of LePT3 under Pi starvation.

### EXAMPLE 3

### Mapping of LePT3: a Pds Candidate

The LePT3 sequence was used as a probe in a Southern blot of genomic DNA from the L. pennellii introgression lines (Zamir and Eshed 1994). The gene mapped to the telomeric portion of chromosome 6, which corresponds to the genetic location of a phosphate deficiency syndrome mutant: Pds. Although this Pds mutant has been available for several years, the controlling gene has remained unknown.

Evidence from by the present study indicates that the novel phosphate transporter gene isolated by this study (SEQ ID NO:1) encodes a novel phosphate transporter protein (SEQ ID NO:2) which is responsible for a substantial portion of the phosphate uptake in plants. This fact is demonstrated by LePT3 mutants which are characterized by phosphate deficiency syndrome.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications cited herein are incorporated by reference in their entirety. Citation or identification of any reference in this application shall not be construed as an admission that such reference is available as prior art to the present invention.

### REFERENCES

1. Bastola D R, Pethe V V, Winicov I (1998) Alfinl, a novel zinc-finger protein in alfalfa roots that binds to promoter elements in the salt-inducible MsPRP2 gene PLANT MOLECULAR BIOLOGY 38: 11293-1135

2. Eshed, Y., and Zamir, D. (1994) Introgression from Lycopersicon pennellii can improve the soluble solid yield of tomato hybrids. Theor. Appl. Genet. 88, 891-897.

3. Gorbunova, V., and Levy, A. A. (1997) Circularized Ac/Ds transposons: formation, structure and fate. Genetics 145, 1161-1169.

4. Hwang I W, Goodman H M (1995) An Arabidopsis-thaliana root-specific kinase homolog is induced by dehydration, ABA, and NaCl. PLANT JOURNAL 8: 37-43

5. Meissner, R., Jacobson, Y., Melamed, S., Levyatuv, S., Shalev, G., Ashri, A., Elkind, Y., and Levy, A. A. (1997) A new model system for Tomato Genetics. Plant J. 12, 1465-1472.

6. Meissner, R. Chague V., Zhu Q., Y. Elkind and A. A. Levy (2000) A high throughput system for transposon tagging and promoter trapping in tomato. Plant J.-accepted

7. Raghothama, K. G. (1999) Phosphate acquisition. Ann. Rev. Plant Physiol. Plant Mol. Biol. 50, 665-693.

8. Rick, C. M., and Yoder, J. I. (1988) Classical and molecular genetics of tomato: highlights and perspectives. Annu Rev. Genet. 22 281-300.

9. Tanksley, S. D. (1993) Linkage map of tomato (Lycopersicon esculentum) (2N=24). In Genetic maps: Locus Maps of Complex Genomes, (J. O'Brien, Eds) Cold Spring Harbor, N.Y.: Cold Spring Harbor Laboratory Press, pp. 6.39-6.60.

What is claimed is:

1. An isolated nucleic acid comprising a genomic, complementary or composite polynucleotide sequence encoding a polypeptide at least 80% homologous SEQ ID NO:2 as determined using the BestFit software of the Wisconsin sequence analysis package, utilizing the Smith and Waterman algorithm, where gap creation penalty equals 8 and gap extension penalty equals 2.

2. The isolated nucleic acid of claim 1, wherein said polypeptide is as set forth in SEQ ID NO:2.

3. The isolated nucleic acid of claim 1, wherein said polypeptide has a phosphate transporter activity.

4. The isolated nucleic acid of claim 1, wherein said polynucleotide is as set forth is SEQ ID NO:1.

5. An isolated nucleic acid comprising a genomic, complementary or composite polynucleotide sequence hybridizable with SEQ ID NO:1 under hybridization conditions of hybridization solution containing 10% dextran sulfate, 1 M NaCl, 1% SDS and 5×10⁶ cpm ³²p labeled probe, at 65° C., with a final wash solution of 1×SSC and 0.1% SDS and final wash at 60° C.

6. An isolated nucleic acid comprising a genomic, complementary or composite polynucleotide sequence at least 65% identical to SEQ ID NO:1 as determined using the BestFit software of the Wisconsin sequence analysis package, utilizing the Smith and Waterman algorithm, where gap weight equals 50, length weight equals 3, average match equals 10 and average mismatch equals −9.

7. An isolated nucleic acid comprising a polynucleotide sequence as set forth in SEQ ID NO:1.

8. An isolated nucleic acid comprising a polynucleotide sequence encoding a polypeptide as set forth in SEQ ID NO: 2.

9. A nucleic acid construct comprising the isolated nucleic acid of claim 1.

10. The nucleic acid construct of claim 9, further comprising a promoter for regulating expression of the isolated nucleic acid.

11. A transformed plant, plant derived tissue or plant cell comprising the nucleic acid construct of claim 9.

12. A pair of oligonucleotides each of at least 17 bases specifically hybridizable with the isolated nucleic acid of claim 1 in an opposite orientation so as to direct exponential amplification of a portion thereof in a nucleic acid amplification reaction.

13. A nucleic acid amplification product obtained using the pair of oligonucleotides of claim 12.

14. A recombinant protein comprising a polypeptide sequence at least 75% homologous to SEQ ID NO:2 as determined using the BestFit software of the Wisconsin sequence analysis package, utilizing the Smith and Waterman algorithm, where gap creation penalty equals 8 and gap extension penalty equals 2.

15. A recombinant protein comprising a polypeptide sequence encoded by a polynucleotide hybridizable with SEQ ID NO:1 under hybridization conditions of hybridization solution containing 10% dextran sulfate, 1 M NaCl, 1% SDS and 5×10⁶ cpm ³²p labeled probe, at 65° C., with a final wash solution of 1×SSC and 0.1% SDS and final wash at 60° C.

16. A recombinant protein comprising a polypeptide sequence encoded by a polynucleotide at least 65% identical with SEQ ID NO:1 as determined using the BestFit software of the Wisconsin sequence analysis package, utilizing the Smith and Waterman algorithm, where gap weight equals 50, length weight equals 3, average match equals 10 and average mismatch equals −9.

17. A recombinant protein comprising a polypeptide sequence as set forth in SEQ ID NO:2.

18. A method of increasing an uptake of phosphate by a plant, the method comprising the step of overexpressing in at least a portion of cells of the plant a phosphate transporter.

19. The method of claim 18, wherein the plant is the family solanaceae.

20. The method of claim 18, wherein the plant is a tomato plant.

21. The method of claim 18, wherein said phosphate transporter is encoded by a polynucleotide at least 65% identical with SEQ ID NO:1 as determined using the BestFit software of the Wisconsin sequence analysis package, utilizing the Smith and Waterman algorithm, where gap weight equals 50, length weight equals 3, average match equals 10 and average mismatch equals −9.

22. The method of claim 21, wherein said polynucleotide is as set forth in SEQ ID NO:1.

23. The method of claim 18, wherein said phosphate transporter is encoded by a polynucleotide hybridizable with SEQ ID NO:1 under hybridization conditions of hybridization solution containing 10% dextran sulfate, 1 M NaCl, 1% SDS and 5×10⁶ cpm ³²p labeled probe, at 65° C., with a final wash solution of 1×SSC and 0.1% SDS and final wash at 60° C.

24. The method of claim 18, wherein said phosphate transporter is at least 80% homologous to SEQ ID NO:2 as determined using the BestFit software of the Wisconsin sequence analysis package, utilizing the Smith and Waterman algorithm, where gap creation penalty equals 8 and gap extension penalty equals 2.

25. An isolated nucleic acid sequence comprising a polynucleotide functional as a plant promoter, wherein said polynucleotide is hybridizable with SEQ ID NO:3 under hybridization conditions of hybridization solution containing 10% dextran sulfate, 1 M NaCl, 1% SDS and 5×10⁶ cpm ³²p labeled probe, at 65° C., with a final wash solution of 1×SSC and 0.1% SDS and final wash at 60° C.

26. The isolated nucleic acid of claim 25, wherein the polynucleotide is as set forth in SEQ ID NO:3.

27. An isolated nucleic acid sequence comprising a polynucleotide functional as a plant promoter, wherein said polynucleotide is at least 50% identical with SEQ ID NO:3 as determined using the BestFit software of the Wisconsin sequence analysis package, utilizing the Smith and Waterman algorithm, where gap weight equals 50, length weight equals 3, average match equals 10 and average mismatch equals −9.

28. A method for effecting phytoremediation of an area polluted with phosphate, the method comprising the steps of:
(a) providing a plant overexpressing a phosphate transporter to thereby facilitate uptake and concentration of phosphate within the plant cells;
(b) planting said plant in the area polluted with phosphate;
(c) following a time period, in which at least a fraction of the phosphate in the area has been accumulated in said plant, harvesting said plant, thereby removing at least said fraction of the phosphate from the area; and optionally
(d) repeating steps (b)-(c) until a sufficient amount of the phosphate has been removed from the area.

29. The method of claim 28, wherein the plant is the family solanaceae.

30. The method of claim 28, wherein the plant is a tomato plant.

31. The method of claim 28, wherein said phosphate transporter is encoded by a polynucleotide at least 65% identical with SEQ ID NO:1 as determined using the BestFit software of the Wisconsin sequence analysis package, utilizing the Smith and Waterman algorithm, where gap weight equals 50, length weight equals 3, average match equals 10 and average mismatch equals −9.

32. The method of claim 31, wherein said polynucleotide is as set forth in SEQ ID NOs:1.

33. The method of claim 28, wherein said phosphate transporter is encoded by a polynucleotide hybridizable with SEQ ID NO:1 hybridization conditions of hybridization solution containing 10% dextran sulfate, 1 M NaCl, 1% SDS and 5×10⁶ cpm ³²p labeled probe, at 65° C., with a final wash solution of 1×SSC and 0.1% SDS and final wash at 60° C.

34. The method of claim 28, wherein said phosphate transporter is at least 80% homologous to SEQ ID NO:2 as determined using the BestFit software of the Wisconsin sequence analysis package, utilizing the Smith and Waterman algorithm, where gap creation penalty equals 8 and gap extension penalty equals 2.

35. The method of claim 28, wherein at least a portion of the phosphate polluting the area is radioactive.

36. A method for generating plants suitable for growth under high phosphate conditions, the method comprising the step of inactivating an expression of an endogenous phosphate transporter gene, said endogenous phosphate transporter gene including a polynucleotide sequence selected from the group consisting of:
(i) at least 65% identical with SEQ ID NO:1 as determined using the BestFit software of the Wisconsin sequence analysis package, utilizing the Smith and Waterman algorithm, where gap weight equals 50, length weight equals 3, average match equals 10 and average mismatch equals −9; and
(ii) hybridizable with SEQ ID NO:1 under hybridization conditions of hybridization solution containing 10% dextran sulfate, 1 M NaCl, 1% SDS and 5×10⁶ cpm ³²p labeled probe, at 65° C., with a final wash solution of 1×SSC and 0.1% SDS and final wash at 60° C.

37. The method of claim 36, wherein said step of inactivating an expression of said phosphate transporter gene is effected by a method selected from the group consisting of:
(i) deleting said endogenous phosphate transporter gene;
(ii) mutating and thereby inactivating said endogenous phosphate transporter gene;
(iii) transcriptionally inactivating said endogenous phosphate transporter gene;
(iv) antisense RNA mediated inactivation of transcripts of said endogenous phosphate transporter gene;
(v) translational inactivation of transcripts of said endogenous phosphate transporter gene; and
(vi) co-suppression of said endogenous phosphate transporter gene via high copy number transformation.


SEQUENCE LISTING (3 sequences)

SEQ ID NO: 1 - DNA, 2099 bp, Lycopersicon esculentum
  misc_feature (1439)..(): any nucleotide
atctcatcaaacacgagaaaatcaaataacatggcctcagacaaccttgtagtgctcaatgcacttgacacagcgcgtacccaatggtaccatgtcactgctgttatcattgctggaatgggatttttcactgatgcatatgatctgttctgtatcactactatctcaaaacttttaggccgtttgtactactacgacccaactacgcatgcccctggaaaattgcctcatgtcgcaaacaactgggtgattggagttgctctagttggtactctatctggccagcttgtttttggttggctcggagacaaacttggtcgaaagaaagtctatggacttacgttaattctcatggtcctttgtgcactttgctcggggttgtcccttgggtatagtgcgaaaggtgtaatagggacactttgtttcttcagattctggcttggatttggaattggaggtgattatcctctttctgctaccatcatgtctgaatacgctaacaaggcaactcgtggggcgttcattgctgctgtttttgctatgcaaggagttgggatcatttttgcagggctggtttcgatgatcatttcaaaattgttcttgatgaggtatgaaggggagccatttaatgtggatgaaattttgtccacggagccccaggcagattatgtttggcggatcgtgttgatgcttggagctcttccagctcttcttacctattattggcgaatgaagatgcctgaaacagggcgttacactgcgattattgaaggaaatgctaaacaagccgcgattaacatggggaaggtacttgatattgaaattcaagcagaaagtgataaattggctcaatttaaagcagctaatgagtattctttactctccaatgagtttttccaacgccacgggcttcatttgattggtacaatgagtacttggttcttgttagacatagctttctatagccaaaacctcacacaaaaggatatatttccagtcatgggactcactagcaacgctaacacaatatccgccttgagggagatgtttgagacatcgcgtgccatgtttgtgattgccttgtttggtacttttcctggttactggttcacagtattcttcattgaaaaaatcggaaggtttagaatacaattgatggggttcttcatgatgtctgttttcatggcgatcattggagtcaaatacgattacctaaagagtaaagaacacaaatggacattcgctactctgtatggtttgactttcttctttgccaattttggccccaattcaaccacatttgtgctccccgcggagctgttccctacaagggtgagatccacttgccacgcgttgagcgcagcttctggtaaggcaggggcaatgatcagtgcatttgggatacaacaatacacacaagatgggaatgttcataaaantcaagacagctatgatactgttggctgttacaaatatggctggattttgttgccgttcttggtgaccgagacaaaagggagatcactcgaagaaattacaggggaggatggcggccagatgagacacagatgaagattagcaaacctgtctgtgtccatcaagacgacgggatgggaatgatacaagttccaaaatttcattgtaatacatcactacgccttgtgcttttctatcttcaattacctctatgaataagagctttaacactatgaatgacctcagtacaacaagttaaattacactaatagtatcgtataaagcctgcatagacatttttatctgttcatgtttacaattttaattcaaacaaggacaaatccatcgtcaattcaatcactacttataattactatgattgtatttgttgaaatattgtttttgctttctaaatctgtatgcaatttgatgccatgtgtaaacattcaaaaggacgcagcagttgcccaatcaagtcaatgattagtcttcattttcaaaccatatatagaaagatacaagtttgaccttatcatgtcttaattgatactgtgcttactaaatttggtgaaactttgacttttctgctgcttgaaaagtgtttttgcctaatttaatttggt

SEQ ID NO: 2 - protein, 513 aa, Lycopersicon esculentum
  misc_feature (470)..(): any amino acid
MASDNLVVLNALDTARTQWYHVTAVIIAGMGFFTDAYDLFCITTISKLLGRLYYYDPTTHAPGKLPHVANNWVIGVALVGTLSGQLVFGWLGDKLGRKKVYGLTLILMVLCALCSGLSLGYSAKGVIGTLCFFRFWLGFGIGGDYPLSATIMSEYANKATRGAFIAAVFAMQGVGIIFAGLVSMIISKLFLMRYEGEPFNVDEILSTEPQADYVWRIVLMLGALPALLTYYWRMKMPETGRYTAIIEGNAKQAAINMGKVLDIEIQAESDKLAQFKAANEYSLLSNEFFQRHGLHLIGTMSTWFLLDIAFYSQNLTQKDIFPVMGLTSNANTISALREMFETSRAMFVIALFGTFPGYWFTVFFIEKIGRFRIQLMGFFMMSVFMAIIGVKYDYLKSKEHKWTFATLYGLTFFFANFGPNSTTFVLPAELFPTRVRSTCHALSAASGKAGAMISAFGIQQYTQDGNVHKXQDSYDTVGCYKYGWILLPFLVTETKGRSLEEITGEDGGQMRHR

SEQ ID NO: 3 - DNA, 150 bp, Lycopersicon esculentum
gcaaagatggaagaaggatattcttcaatggtttatcagggaatttcttgttctagatacagaaactaggcttaaatgtcaattaattgatcccataactgtctatatatacgtaaattgagcaatttagagatgatcatacatcaacca